(19) United States
(12) Patent Application Publication
(10) Pub. No.: US 2007/0027530 A1
(43) Pub. Date: Feb. 1, 2007

(54) Intraluminal device, catheter assembly, and method of use thereof

(75) Inventors: Sean T. Saint, San Diego, CA (US); John E. Nolting, Poway, CA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 11/189,415

(22) Filed: Jul. 26, 2005

(51) Int. Cl. A61F 2/06

(52) U.S. Cl. 623/1.42

(57) ABSTRACT

An intraluminal device, an intraluminal stent delivery system, and a method of manufacturing the same. The device comprises a body including at least one strut. The strut comprises at least one raised portion positioned on an outer surface of the body. At least one therapeutic agent coating is positioned on the strut. The therapeutic agent coating comprises an effective therapeutic agent coating area greater than about 50 percent of a strut total surface area. The stent delivery system includes a catheter and the stent disposed on the catheter. The manufacturing method includes providing a body including at least one strut. The strut comprises at least one raised portion positioned on an outer surface of the body. At least one therapeutic agent coating is applied on the strut to an effective therapeutic agent coating area greater than about 50 percent of a strut total surface area.

### TECHNICAL FIELD OF THE INVENTION

The present invention relates generally to the field of implantable medical devices. More particularly, the invention relates to an intraluminal device, catheter assembly, and method of use thereof.

### BACKGROUND OF THE INVENTION

Balloon angioplasty has been used for the treatment of narrowed and occluded blood vessels. A frequent complication associated with the procedure is restenosis, or vessel re-narrowing. Within 3-6 months of angioplasty an unacceptably high degree and incidence of restenosis occurs. To reduce the incidence of re-narrowing, several strategies have been developed. Implantable devices, such as stents, have been used to reduce the rate of angioplasty related restenosis by about half. The use of such intraluminal devices has greatly improved the prognosis of these patients. Nevertheless, restenosis remains a formidable problem associated with the treatment of narrowed blood vessels.

Restenosis associated with interventional procedures such as balloon angioplasty may occur by at least two mechanisms: thrombosis and intimal hyperplasia. During angioplasty, a balloon is inflated within an affected vessel thereby compressing the blockage and imparting a significant force, and subsequent trauma, upon the vessel wall. The natural antithrombogenic lining of the vessel lumen may become damaged thereby exposing thrombogenic cellular components, such as matrix proteins. The cellular components, along with the generally antithrombogenic nature of any implanted materials (e.g., a stent), may lead to the formation of a thrombus, or blood clot. The risk of thrombosis is generally greatest immediately after the angioplasty.

A second mechanism of restenosis is intimal hyperplasia, or excessive tissue re-growth. The trauma imparted upon the vessel wall from the angioplasty is generally believed to be an important factor contributing to hyperplasia. This exuberant cellular growth may lead to vessel “scarring” and significant restenosis. The risk of hyperplasia associated restenosis is usually greatest 3 to 6 months after the procedure.

Prosthetic devices, such as stents or grafts, may be implanted during interventional procedures such as balloon angioplasty to reduce the incidence of vessel restenosis. To improve device effectiveness, stents may be coated with one or more therapeutic agents providing a mode of localized drug delivery. The therapeutic agents are typically intended to limit or prevent the aforementioned mechanisms of restenosis. For example, antithrombogenic agents such as heparin or clotting cascade IIb/IIIa inhibitors (e.g., abciximab and eptifibatide) may be coated on the stent thereby diminishing thrombus formation. Such agents may effectively limit clot formation at or near the implanted device. Some antithrombogenic agents, however, may not be effective against intimal hyperplasia. Therefore, the stent may also be coated with antiproliferative agents or other compounds to reduce excessive endothelial re-growth. Therapeutic agents provided as coating layers on implantable medical devices may effectively limit restenosis and reduce the need for repeated treatments.

Several strategies have been developed for coating one or more therapeutic agents onto the surface of medical devices, such as stents. Standard methods may include dip coating, spray coating, and chemical bonding. The coating may be applied as a mixture, solution, or suspension of polymeric material and/or therapeutic agent dispersed in an organic vehicle or a solution or partial solution. Further, the coating may include one or more sequentially applied, relatively thin layers deposited in a relatively rapid sequence. The stent is typically in a radially expanded state during the application process. In some applications, the coating may include a composite initial tie coat, or undercoat, and a composite topcoat, or cap coat. The coating thickness ratio of the topcoat to the undercoat may vary with the desired effect and/or the elution system.

Once the medical device is coating with therapeutic agent(s), the coating (including any undercoat and/or cap coat) may be smeared, rubbed off, or even (partially) removed should the device come into contact with another surface. To illustrate, a self-expandable stent may be contained within a protective sheath before and during deployment within a blood vessel. Once properly positioned, the sheath may be retracted thereby allowing the stent to expand. Prior to and during the deployment procedure, the stent may come into contact with the sheath thereby compromising the integrity of the coating. Further, the stent may contact the blood vessel surface further disrupting the coating. As such, it would be desirable to provide a strategy for maintaining the integrity of a medical device coating.

Accordingly, it would be desirable to provide an intraluminal device, catheter assembly, and method of use thereof that would overcome the aforementioned and other limitations.

### SUMMARY OF THE INVENTION

A first aspect according to the invention provides an intraluminal device. The device comprises a body including at least one strut. The strut comprises at least one raised portion positioned on an outer surface of the body. At least one therapeutic agent coating is positioned on the strut. The therapeutic agent coating comprises an effective therapeutic agent coating area greater than about 50 percent of a strut total surface area.

A second aspect according to the invention provides an intraluminal stent delivery system. The system comprises a catheter and a stent disposed on the catheter. The stent comprises a body including at least one strut. The strut comprises at least one raised portion positioned on an outer surface of the body. At least one therapeutic agent coating is positioned on the strut. The therapeutic agent coating comprises an effective therapeutic agent coating area greater than about 50 percent of a strut total surface area.

A third aspect according to the invention provides a method of manufacturing an intraluminal device. The method includes providing a body including at least one strut. The strut comprises at least one raised portion positioned on an outer surface of the body. At least one therapeutic agent coating is applied on the strut to an effective therapeutic agent coating area greater than about 50 percent of a strut total surface area.

The foregoing and other features and advantages of the invention will become further apparent from the following description of the presently preferred embodiments, read in conjunction with the accompanying drawings. The detailed description and drawings are merely illustrative of the invention, rather than limiting the scope of the invention being defined by the appended claims and equivalents thereof.

### BRIEF DESCRIPTION OF THE DRAWINGS

FIG. 1 is a perspective view of an intraluminal stent delivery system including a stent mounted on a balloon, in accordance with one embodiment of the present invention;

FIG. 2 is a detailed view of the stent of FIG. 1;

FIG. 3 is a cross-sectional view of the stent of FIG. 1; and

FIGS. 4A, 4B, and 4C are temporal views of a portion of the stent of FIG. 1 shown in cross-section and positioned adjacent a vessel wall, in accordance with the present invention.

### DESCRIPTION OF THE PRESENTLY PREFERRED EMBODIMENTS

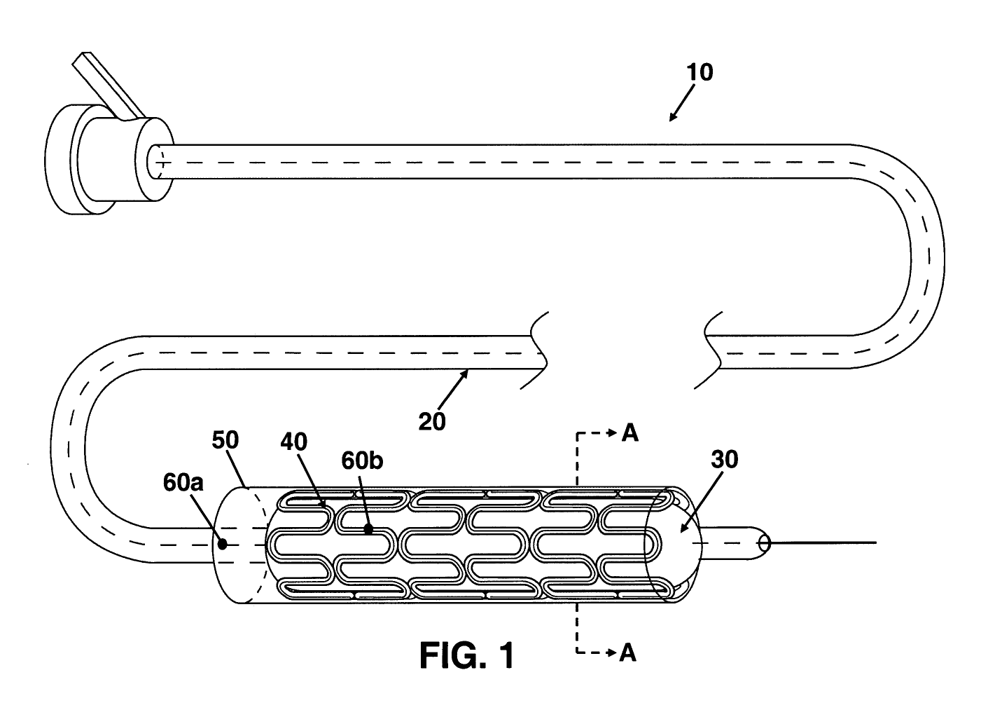

Referring to the drawings, which are not necessarily drawn to scale and wherein like reference numerals refer to like elements, FIG. 1 is a perspective view of an intraluminal stent delivery system in accordance with one embodiment of the present invention and shown generally by numeral 10. System 10 may include a catheter 20, a balloon 30 operably attached to the catheter 20, and a stent 40 disposed on the balloon 30. Balloon 30 may be any variety of balloons capable of expanding the stent 40. Balloon 30 may be manufactured from any sufficiently elastic material such as polyethylene, polyethylene terephthalate (PET), nylon, or the like. Stent 40 may be expanded with the balloon 30. System 10 may include a sheath 50 to retain the stent 40 in a collapsed state and to prevent contact with surfaces, such as a vessel wall, during advancement through a vessel lumen and subsequent deployment. Once the stent 40 is properly positioned, the sheath 50 may be retracted thereby allowing the stent to assume its expanded shape. The advancement, positioning, and deployment of stents and similar devices are well known in the art.

The terms “catheter” and “stent”, as used herein, may include any number of intravascular and/or implantable prosthetic devices (e.g., a stent-graft); the examples provided herein are not intended to represent the entire myriad of devices that may be adapted for use with the present invention. Although the devices are described herein are primarily done so in the context of deployment within a blood vessel, it should be appreciated that intravascular and/or implantable prosthetic devices in accordance with the present invention may be deployed in other vessels, such as a bile duct, intestinal tract, esophagus, airway, etc.

Catheter 20 comprises an elongated tubular member manufactured from one or more polymeric materials, sometimes in combination with metallic reinforcement. In some applications (such as smaller, more tortuous arteries), it is desirable to construct the catheter from very flexible materials to facilitate advancement into intricate access locations. Numerous over-the-wire, rapid-exchange, and other catheter designs are known and may be adapted for use with the present invention. The stent, particularly the self-expanding variety, may be positioned within a sheath to retain the stent in the collapsed state until it is at the deployment site. The sheath may then be retracted thereby allowing the self-expanding to assume its naturally expanded shape. The sheath may also function to prevent the stent from inadvertent contact with other surfaces to, for example, prevent injury to a vessel wall or to maintain the integrity of a therapeutic agent coated on the stent. Self-expanding stents typically do not require a balloon to provide the radial forces needed to expand the stent. However, the balloon may provide other advantages such as ensuring proper placement of the stent within the vessel (i.e., to prevent the stent from slipping or ‘jumping’ due to its inherent spring-like properties).

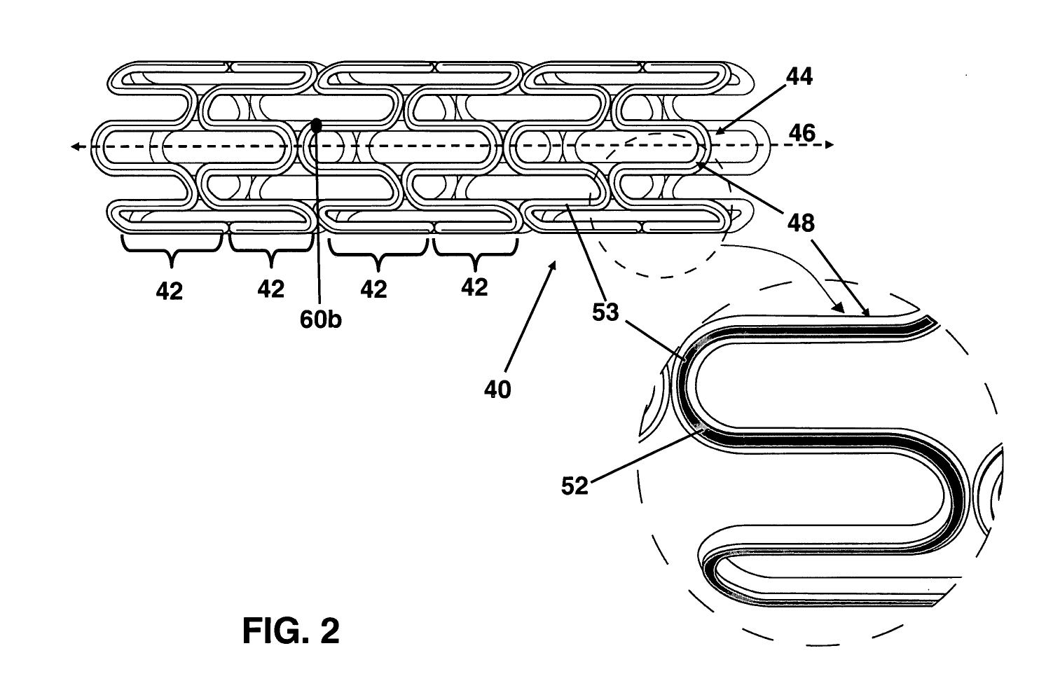

FIG. 2 is a detailed view of the stent 40 shown in FIG. 1. Stent 40 may be of any variety of implantable prosthetic devices as known in the art. Stent 40 may be manufactured from a skeletal framework or mesh of material forming a tube-like structure and may be capable of self-expanding or being expanded by another device such as a balloon or other means. In one embodiment, the stent 40 may include a plurality of identical cylindrical stent segments 42 placed end to end. Those skilled in the art will recognize that the number of stent segments may vary and that numerous other stents, stent-grafts, and implantable prosthetic devices may be adapted for use with the present invention; the described stent 40 is provided merely as an example.

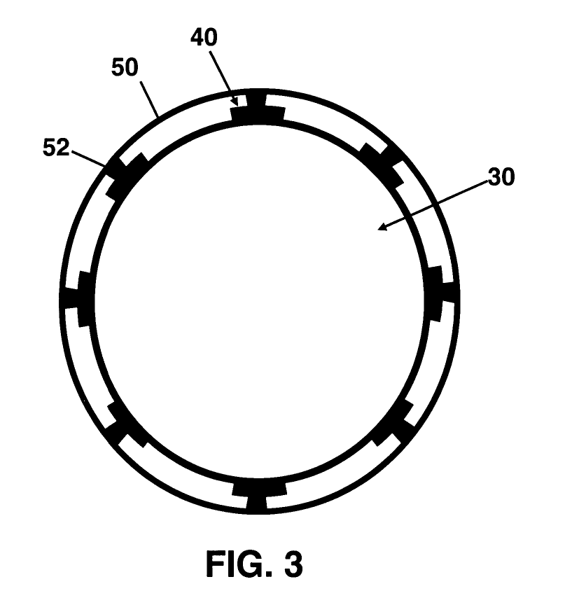

In one embodiment, the stent 40 includes a generally tubular body 44 defining a passageway extending along a longitudinal axis 46. Stent 40 may include the plurality of cylindrical segments 42 arranged successively along the longitudinal axis 46. Each of the cylindrical segments 42 may have a length along the longitudinal axis 46 and may be comprised of at least one strut 48, which in this case are generally U-shaped. Struts 48 may open in alternating directions along the longitudinal axis 46 about the perimeter or circumference of the cylindrical segments 42. Struts 48 include at least one raised portion 52 positioned on an outer surface 53 of the body 44. The raised portion 52 may space the outer surface 53 of the body 44 substantially away from an inner surface of the sheath 50 or any other surfaces that may contact the stent 60. The space provides a buffer between surfaces, such as the sheath 50 or a vessel wall, which may contact the outer surface 53 of the body 44. As such, the integrity of therapeutic agent coating(s) provided on the stent 40 may be maintained. FIG. 3 is a cross-sectional view of the stent 40 positioned within the sheath 50, taken along lines A-A of FIG. 1, illustrating spacing of the stent 60 from the sheath 50 provided by the raised portion 52.

Referring again to FIG. 2, the stent 40 preferably is compressed into a smaller diameter (i.e., when “loaded” on the balloon and/or within the sheath 50) for deployment within a vessel lumen at which point the stent 40 may be expanded to provide support to the vessel. Once properly positioned within a vessel lumen, the sheath 50 is retracted as the balloon 30 and stent 40 expand. Cylindrical segments 42 may move radially outward from the longitudinal axis 46 as the stent 40 expands. At least one (radiopaque) marker 60a, 60b may be disposed on the stent 40, catheter 20, and or component thereof to allow in situ visualization and proper advancement, positioning, and deployment of the stent 40. The marker(s) may be manufactured from a number of materials used for visualization in the art including radiopaque materials platinum, gold, tungsten, metal, metal alloy, and the like. Marker(s) may be visualized by fluoroscopy, IVUS, and other methods known in the art. Those skilled in the art will recognize that numerous devices and methodologies may be utilized for deploying a stent and other intraluminal device in accordance with the present invention.

In one embodiment, the stent 40 may be expanded by a balloon or some other means of providing radial forces. Stent 40 may be manufactured from an inert, biocompatible material with high corrosion resistance. The biocompatible material should ideally be plastically deformed at low-moderate stress levels. In another embodiment, the stent 40 may be of the self-expanding variety and manufactured from, for example, a nickel titanium alloy and/or other alloy(s) that exhibit superlastic behavior (i.e., capable of significant distortion without plastic deformation). Suitable materials for stents include, but are not limited to, tantalum, stainless steel, titanium ASTM F63-83 Grade 1, niobium, high carat gold K 19-22, and MP35N. Furthermore, the stent material may include any number of other metallic and/or polymeric biocompatible materials recognized in the art for such devices.

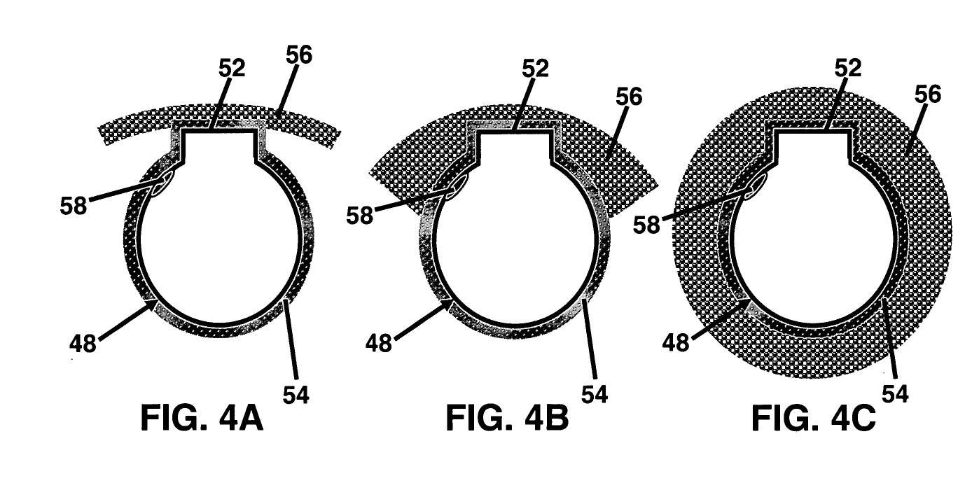

As shown in cross-sectional views provided in FIGS. 4A, 4B, and 4C, the strut 48 includes at least one therapeutic agent coating 54 positioned thereon. Strut 48 is shown positioned adjacent a vessel wall 56. After the stent is deployed, as shown in FIG. 4A, the vessel wall 46 primarily contacts the raised portion 52 of the strut 48. After some time has passed, as shown in FIG. 4B, endothelium of the vessel wall 56 may begin to encompass the strut 48 (i.e., by the process of the progressive growth of the vessel endothelium) thereby contacting a greater proportion of a strut 48 total surface area. After more time, as shown in FIG. 4C, the endothelium may substantially encompass the entire strut 48 thereby contacting the virtually all of the strut 48 total surface area. Therapeutic agent coating 54 may effectively deliver the therapeutic agent(s) to the vessel wall 56 in relation to 1) the proportion of the strut 48 total surface area that is coated and 2) the proportion of the strut 48 total surface area that contacts the vessel wall 56. These two properties define an effective therapeutic agent coating area of the stent 40. Preferably, the coating 54 comprises an effective therapeutic agent coating area greater than about 50 percent of the strut 48 total surface area. As illustrated and described herein, the effective therapeutic agent coating area comprises about 100 percent of the strut 48 total surface area.

In one embodiment, the strut 48 may include one or more recessed area(s) 58 formed therein. Recessed area 58 preferably includes the therapeutic agent coating 54 positioned therein. Recessed area 58 provides a reservoir of therapeutic agent so that certain portions of the strut 48 may provide additional or a different type of drug elution. Those skilled in the art will recognize that the recessed area may take the form of various pits, channels, geometries, sizes, and include a variety of therapeutic agent(s) positioned therein.

In one embodiment, the therapeutic agent may comprise one or more drugs, polymers, a component thereof, a combination thereof, and the like. For example, the therapeutic agent may include a mixture of a drug and a polymer as known in the art. Some exemplary drug classes that may be included are antiangiogenesis agents, antiendothelin agents, antimitogenic factors, antioxidants, antiplatelet agents, antiproliferative agents, antisense oligonucleotides, antithrombogenic agents, calcium channel blockers, clot dissolving enzymes, growth factors, growth factor inhibitors, nitrates, nitric oxide releasing agents, vasodilators, virus-mediated gene transfer agents, agents having a desirable therapeutic application, and the like. Specific example of drugs include abciximab, angiopeptin, colchicine, eptifibatide, heparin, hirudin, lovastatin, methotrexate, streptokinase, taxol, ticlopidine, tissue plasminogen activator, trapidil, urokinase, and growth factors VEGF, TGF-beta, IGF, PDGF, and FGF.

The polymer generally provides a matrix for incorporating the drug within the coating, or may provide means for slowing the elution of an underlying therapeutic agent when it comprises a cap coat. Some exemplary biodegradable polymers that may be adapted for use with the present invention include, but are not limited to, polycaprolactone, polylactide, polyglycolide, polyorthoesters, polyanhydrides, poly(amides), poly(alkyl-2-cyanocrylates), poly(dihydropyrans), poly(acetals), poly(phosphazenes), poly(dioxinones), trimethylene carbonate, polyhydroxybutyrate, polyhydroxyvalerate, their copolymers, blends, and copolymers blends, combinations thereof, and the like. Exemplary non-biodegradable polymers that may be adapted for use with the present invention may be divided into at least two classes. The first class includes hydrophobic polymers such as polyolefins, acrylate polymers, vinyl polymers, styrene polymers, polyurethanes, polyesters, epoxy, nature polymers, their copolymers, blends, and copolymer blends, combinations thereof, and the like. The second class includes hydrophilic polymers, or hydrogels, such as polyacrylic acid, polyvinyl alcohol, poly(N-vinylpyrrolidone), poly(hydroxy-alkylmethacrylate), polyethylene oxide, their copolymers, blends and copolymer blends, combinations of the above, and the like.

Solvents are typically used to dissolve the therapeutic agent and polymer to comprise a therapeutic agent coating solution. Some exemplary solvents that may be adapted for use with the present invention include, but are not limited to, acetone, ethyl acetate, tetrahydrofuran (THF), chloroform, N-methylpyrrolidone (NMP), and the like.

Those skilled in the art will recognize that the nature of the drug and polymer may vary greatly and are typically formulated to achieve a given therapeutic effect, such as limiting restenosis, thrombus formation, hyperplasia, etc. Once formulated, a therapeutic agent solution (mixture) comprising the coating 54 may be applied to the stent 40 by any of numerous strategies known in the art including, but not limited to, spraying, dipping, rolling, nozzle injection, and the like. It will be recognized that the coating 54 may be alternatively layered, arranged, configured on/within the stent 40 depending on the desired effect. Once applied, the coatings 54 may be dried (i.e., by allowing the solvent to evaporate) and, optionally, other coatings (e.g., a “cap” coat) added thereon. Numerous strategies of applying the coating 54 in accordance with the present invention are known in the art. The coating 54 and additional polymer(s) may be applied simultaneously or separately by, for example, differentially masking the stent 40. The inventors contemplate numerous strategies for applying the coating 54 as would be appreciated by one skilled in the art.

While the embodiments of the invention disclosed herein are presently considered to be preferred, various changes and modifications can be made without departing from the spirit and scope of the invention. For example, the intraluminal device is not limited to any particular design, such as a stent. In addition, the therapeutic agent composition and coating process may be varied considerably while providing a multi-order elution kinetic.

Upon reading the specification and reviewing the drawings hereof, it will become immediately obvious to those skilled in the art that myriad other embodiments of the present invention are possible, and that such embodiments are contemplated and fall within the scope of the presently claimed invention. The scope of the invention is indicated in the appended claims, and all changes that come within the meaning and range of equivalents are intended to be embraced therein.

1. An intraluminal device comprising:
a body including at least one strut, the strut comprising at least one raised portion positioned on an outer surface of the body;
at least one therapeutic agent coating positioned on the strut, the therapeutic agent coating comprising an effective therapeutic agent coating area greater than about 50 percent of a strut total surface area.

2. The device of claim 1 wherein the body is selected from a group consisting of a stent and a graft.

3. The device of claim 1 wherein the strut comprises at least one recessed area formed therein, the at least one recessed area including the therapeutic agent coating positioned therein.

4. The device of claim 1 wherein the strut total surface area comprises the outer surface of the body and an inner surface of the body.

5. The device of claim 1 wherein the at least one strut is substantially encompassed by a vessel endothelium.

6. The device of claim 1 further comprising a sheath including a lumen formed therein for receiving the body.

7. The device of claim 6 wherein the at least one raised portion spaces the outer surface of the body substantially away from a sheath inner surface of the sheath.

8. An intraluminal stent delivery system comprising:
a catheter; and
a stent disposed on the catheter, the stent comprising a body including at least one strut, the strut comprising at least one raised portion positioned on an outer surface of the body;
at least one therapeutic agent coating positioned on the strut, the therapeutic agent coating comprising an effective therapeutic agent coating area greater than about 50 percent of a strut total surface area.

9. The system of claim 8 wherein the strut comprises at least one recessed area formed therein, the at least one recessed area including the therapeutic agent coating positioned therein.

10. The system of claim 8 wherein the strut total surface area comprises the outer surface of the body and an inner surface of the body.

11. The system of claim 8 wherein the at least one strut is substantially encompassed by a vessel endothelium.

12. The system of claim 8 further comprising a sheath including a lumen formed therein for receiving the body.

13. The system of claim 12 wherein the at least one raised portion spaces the outer surface of the body substantially away from a sheath inner surface of the sheath.

14. A method of manufacturing an intraluminal device, the method comprising:
providing a body including at least one strut, the strut comprising at least one raised portion positioned on an outer surface of the body;
applying at least one therapeutic agent coating on the strut to an effective therapeutic agent coating area greater than about 50 percent of a strut total surface area.

15. The method of claim 14 wherein the body is selected from a group consisting of a stent and a graft.

16. The method of claim 14 wherein the at least one therapeutic agent coating is applied to at least one recessed area formed within the strut.

17. The method of claim 14 wherein the strut total surface area comprises the outer surface of the body and an inner surface of the body.

18. The method of claim 14 further comprising substantially encompassing the at least one strut with a vessel endothelium.

19. The method of claim 14 further comprising positioning the body within a lumen formed within a sheath.

20. The method of claim 19 further comprising spacing the outer surface of the body substantially away from a sheath inner surface of the sheath.
